(19) United States
(12) Patent Application Publication
(10) Pub. No.: US 2014/0024093 A1
(43) Pub. Date: Jan. 23, 2014

(54) PROCESS FOR THE PRODUCTION OF DIGESTED BIOMASS USEFUL FOR CHEMICALS AND BIOFUELS

(71) Applicant: SHELL OIL COMPANY, Houston, TX (US)

(72) Inventors: Robert Lawrence Blackbourn, Houston, TX (US); Juben Nemchand Chheda, Houston, TX (US); Evert Van Der Heide, Amsterdam (NL); Paul Richard Weider, Houston, TX (US)

(73) Assignee: SHELL OIL COMPANY

(21) Appl. No.: 13/851,357

(22) Filed: Jul. 25, 2013

Related U.S. Application Data
(60) Provisional application No. 61/617,208, filed on Mar. 29, 2012.

(51) Int. Cl. D21C 3/20; C12P 7/18

(52) U.S. Cl. CPC D21C 3/20; C12P 7/18
USPC 435/158; 162/76

(57) ABSTRACT

In the pretreatment, the biomass is contacted with a solution containing at least one α-hydroxysulfonic acid thereby at least partially hydrolyzing the biomass to produce a pretreated stream containing a solution that contains at least a portion of hemicelluloses and a residual biomass that contains celluloses and lignin; separating at least a portion of the solution from the residual biomass providing an solution stream and a pretreated biomass stream; then contacting the pretreated biomass stream with a cooking liquor containing at least one alkali selected from the group consisting of sodium hydroxide, sodium carbonate, sodium sulfide, potassium hydroxide, potassium carbonate, ammonium hydroxide, and mixtures thereof and water. A process that allows for higher recovery of carbohydrates and thereby increased yields is provided. Alcohols useful as fuel compositions are also produced from biomass by pretreating the biomass prior to hydrolysis and fermentation.

### CROSS REFERENCE TO RELATED APPLICATIONS

This application claims the benefit of U.S. Provisional Application No. 61/617,208, filed on Mar. 29, 2012, the disclosure of which is incorporated herein by reference in its entirety.

### FIELD OF THE INVENTION

Embodiments of this invention relate to a process for the production of alcohols from cellulosic biomass.

### BACKGROUND

This section is intended to introduce various aspects of the art, which may be associated with exemplary embodiments of the present invention. This discussion is believed to assist in providing a framework to facilitate a better understanding of particular aspects of the present invention. Accordingly, it should be understood that this section should be read in this light, and not necessarily as admissions of any prior art.

The basic feedstocks for the production of first generation biofuels are often seeds, like grains such as wheat and corn, that produce starch or sugar cane and sugar beets that produce sugars that is fermented into bioethanol. However, the production of ethanol from these feedstocks suffers from the limitation that much of the farmland which is suitable for their production is already in use for food production.

Biologically produced alcohols, most commonly ethanol, and less commonly propanol and butanol, can be produced by the action of enzymes and microorganisms through the hydrolysis of starches or celluloses to glucose and subsequently fermentation of sugars. Cellulosic ethanol production uses non-food crops and does not divert food away from the food chain or inedible waste products which does not change the area of farmland in use for food products. However, production of ethanol from cellulose poses a difficult technical problem. Some of the factors for this difficulty are the physical density of lignocelluloses (like wood) that can make penetration of the biomass structure of lignocelluloses with chemicals difficult and the chemical complexity of lignocelluloses that lead to difficulty in breaking down the long chain polymeric structure of cellulose into sugars that can be fermented. Thus, it requires a great amount of processing to make the sugar monomers available to the microorganisms that are typically used to produce ethanol by fermentation.

Lignocellulose is the most abundant plant material resource and is composed mainly of cellulose, hemicelluloses and lignin. Woodchips are used in pulp and paper mills to convert wood into wood pulp by chemical or physical processes, usually Kraft process. In a Kraft process, woodchips are treated in a digester with a mixture of sodium hydroxide and sodium sulfide, known as white liquor. The woodchips are impregnated with a cooking solution that contains white liquor. White liquor is produced in the chemical recovery process.

In a continuous digester, the materials are fed at a rate which allows the pulping reaction to be complete by the time the materials exit the reactor. Typically, delignification requires several hours at about 155° C. to 175° C., typically around 170° C. Under these conditions lignin and some hemicelluloses degrade to give fragments that are soluble in the strongly basic white liquor. The solid pulp (about 50% by weight based on the dry wood chips) known as brown stock is collected and washed to produce brownstock pulp that typically contains 3% to 4% by weight lignin (Kappa #20-30) for softwood and 2% to 3% by weight lignin (Kappa #10-20) for hardwood, which is further passed through a series of bleaching steps to generate paper-quality pulp. The combined liquids known as black liquor contains extracted lignins, carbohydrates, sodium hydroxide, sodium sulfide and other inorganic salts. The black liquor is at about 15% solids and is concentrated in a multiple effect evaporator to 60% or even 75% solids and burned in the recovery boiler to recover the inorganic chemicals for reuse in the process. The combustion is carried out such that sodium sulfate, added as make-up is reduced to sodium sulfide by the organic carbon in the mixture. The molten salts from the recovery boiler are dissolved in process water known as “weak white liquor” composed of all liquors used to wash lime mud and green liquor precipitates. The resulting solution of sodium carbonate and sodium sulfide is known as “green liquor.” Green liquor contains at least 4 wt %, typically 5 wt %, of sodium carbonate concentration. Green liquor is mixed with calcium hydroxide to regenerate the white liquor used in the pulping process.

Currently there exist two broad categories of processes for the hydrolysis of cellulose. One category uses mineral acids such as sulfuric acid as discussed in U.S. Pat. No. 5,726,046, while the second category uses enzymes. The mineral acid most commonly used in mineral acid process is sulfuric acid. In general sulfuric acid hydrolysis can be categorized as either dilute acid hydrolysis or concentrated acid hydrolysis.

The dilute acid processes generally involve the use of about 0.5% to 15% sulfuric acid to hydrolyze the cellulosic material. In addition, temperatures ranging from about 90° C. to 600° C., and pressure up to 800 psi are necessary to affect the hydrolysis. At high temperatures, the sugars degrade to form furfural and other undesirable by-products. The resulting fermentable sugar yields are generally low, less than 50% and process equipment must be employed to physically remove furfural before further processing.

The concentrated acid processes have been somewhat more successful, producing higher yields of sugar. However, these processes typically involve the use of about 60% to 90% sulfuric acid to affect hydrolysis, leading to high cost due to the cost of handling concentrated sulfuric acid and it subsequent recovery.

The additional problems faced in the acid hydrolysis processes include the production of large amounts of gypsum when the spent or used acid is neutralized. The low sugar concentrations resulting from the processes require the need for concentration before fermentation can proceed. When hydrolysis is carried out at temperatures above 150° C., compounds such as furfural are produced from the degradation of pentoses. These compounds inhibit fermentation, and some may be toxic. Furthermore, the degradation of pentose sugars results in a loss of yield.

U.S. Pat. No. 4,070,232 describes the prehydrolysis step in the presence of dilute acid solutions containing a mixture of HCl, formic and acetic acid which is pretty corrosive mixture requiring expensive process equipment. Also, the recovery of hemicelluloses is low due to short residence times (about 7-20 minutes) at low temperatures (about 100-130° C.).

U.S. Application Publication No. 2008/0190013 describes use of ionic liquids to pretreat lgnocellulosic material. However, ionic liquids are generally more expensive and difficult to recover, while cleaning (building-up of heavy components) is required. Minor losses will make the process uneconomical.

### SUMMARY

Accordingly, in one embodiment, there is provided a process for producing a digested biomass stream comprising:
(a) providing a biomass containing celluloses, hemicelluloses and lignin;
(b) producing a pretreated stream by contacting the biomass with a solution containing at least one α-hydroxysulfonic acid at a temperature of about 150° C. or less, wherein the pretreated stream comprises a solution comprising at least a portion of hemicelluloses and a residual biomass comprising celluloses and lignin;
(c) providing a solution stream and a pretreated biomass stream by separating at least a portion of the solution from the residual biomass;
(d) providing a digested biomass stream and a chemical liquor stream by contacting the pretreated biomass stream with a cooking liquor comprising (i) about 0.5 wt % to about 20 wt %, based on the cooking liquor, (ii) at least one alkali selected from the group consisting of sodium hydroxide, sodium carbonate, sodium sulfide, potassium hydroxide, potassium carbonate, ammonium hydroxide, and any combination thereof, (iii) water, at a biomass to cooking liquor ratio in a range of 2 to 6, at a temperature in a range from about 60° C. to about 230° C., wherein the digested biomass stream comprises digested biomass containing cellulosic material, hemicellulosic material, and at least a portion of lignin, and the chemical liquor stream comprises at least a portion of lignin and at least one sodium compound, potassium compound, or ammonium compound; and
(e) removing at least a portion of lignin and hemicellulosic material in the digested biomass stream and producing lignin-removed digested biomass stream by washing the digested biomass stream with a water stream.

In another embodiment, the process further comprises removing the α-hydroxysulfonic acid from the solution stream by heating and/or reducing pressure to produce an acid-removed product substantially free of the α-hydroxysulfonic acid and recycling the removed α-hydroxysulfonic acid to step (b) as components or in recombined form.

In yet another embodiment, the process further comprises: producing a hydrolyzate containing from about 4% to 30% by weight of fermentable sugar by contacting the lignin-removed biomass stream with an enzyme solution comprising cellulases and optionally xylanases at a pH in a range from about 3 to about 7 at a temperature in a range from about 30° C. to about 90° C.; producing an alcohol stream containing at least one alcohol having 2 to 18 carbon atoms by fermenting the hydrolyzate in the presence of a microorganism at a temperature in a range from about 25° C. to about 55° C. at a pH in a range from about 4 to about 6; and recovering at least one of said alcohol from the alcohol stream.

Advantages and other features of embodiments of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows a block schematic diagram illustrating one embodiment of the biomass digestion process.

FIG. 2 shows a block schematic diagram illustrating another embodiment of the process.

FIG. 3 shows a block schematic diagram illustrating yet another embodiment of the process.

FIG. 4 shows a portion of a block schematic diagram illustrating yet another embodiment of the process.

### DETAILED DESCRIPTION OF PREFERRED EMBODIMENTS

It has now been found that by improving the digestion of biomass treatment and subsequent processing of such digested product, a process with high yield production of chemicals and alcohol suitable for use in fuels can be obtained. Embodiments described herein have significant benefits over other biomass pretreatments wherein the toxic components such as furfural and acetic acid are essentially eliminated for the fermentation process. Also, bulk removal of lignin allows improved mass transfer of enzymes to cellulose for conversion to fermentable sugars.

In some embodiments, the systems for performing certain aspects of the presently disclosed methods can be configured by repurposing the components of a pulp mill that previously used the Kraft pulping process. Such repurposing can allow for the employment of the presently disclosed methods with relatively low capital investment compared to many other proposed biomass-to-ethanol methods. Further, the control objective in a typical Kraft pulping is to cook to a target kappa number to correspond to lignin content of less than 4%. (see Handbook for Pulp & Paper Technologists, published in 2002 by Angus Wilde Publications Inc., Vancouver, B.C.). In some embodiments, the entire pretreatment-digestion process is conducted under conditions that produce lignin content of about 1% to about 20%, preferably about 5% to about 18%, which is then further processed in a manner to produce alcohol. It has been found that a process can be obtained to produce sugars and alcohols in high yields from biomass containing cellulosic fibers.

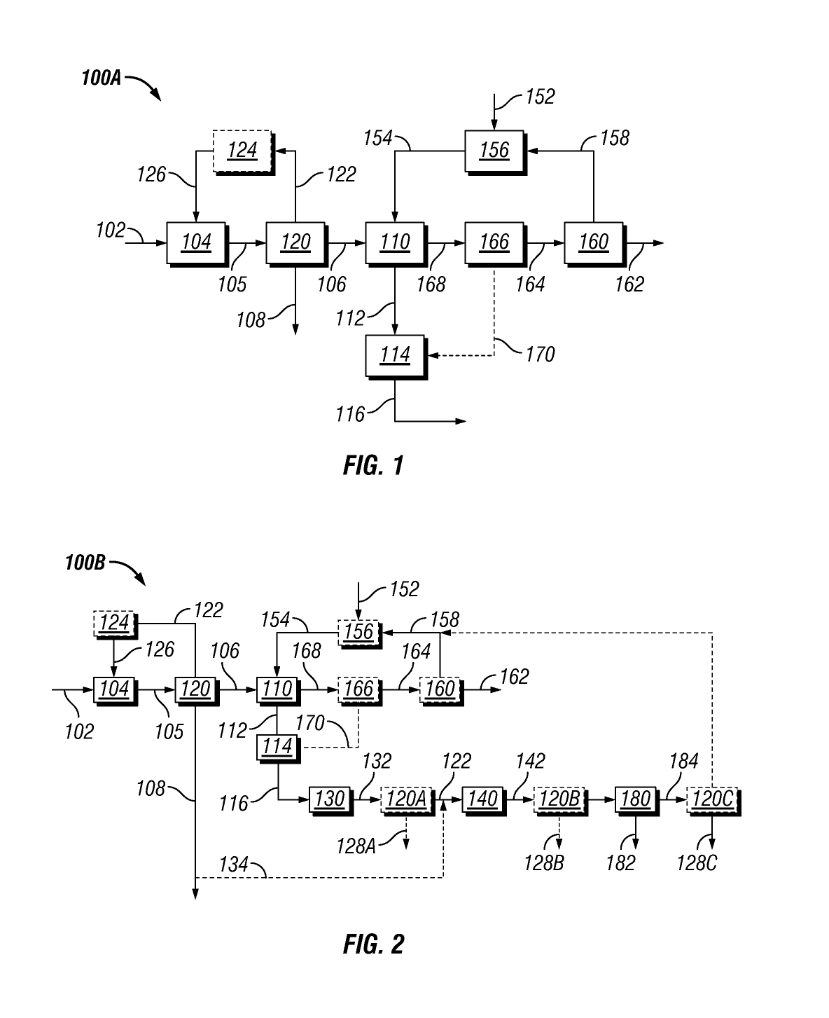

In reference to FIG. 1, in one embodiment of the invention process 100A, biomass 102 is provided to a pretreatment system 104 that may have one or more vessels, where the biomass is contacted with a solution containing at least one α-hydroxysulfonic acid to produce a product stream 105 containing hemicelluloses in solution and a residual biomass containing celluloses and lignin. At least a portion of the solution is separated, in a separation system (and/or acid removal system) 120, from the residual biomass providing a solution stream 108 and a pretreated biomass stream 106. The pretreatment system 104 can comprise a number of components including in situ generated α-hydroxysulfonic acid. The term “in situ” as used herein refers to a component that is produced within the overall process; it is not limited to a particular reactor for production or use and is therefore synonymous with an in process generated component. Optionally the reacted product stream from 104 is introduced to acid removal system 120 where the acid is recovered 122 (and optionally scrubbed 124) and recycled via recycle stream 126 to 104 and product stream 106 containing at least one fermentable sugar (e.g., pentose and optionally hexose) substantially free of the alpha-hydroxysulfonic acids is produced for further processing. The pretreated biomass stream 106 is contacted, in a digestion system 110 that may have one or more digester(s), with a cooking liquor (optionally via cooking liquor feed stream 154) that was optionally at least a portion recycled from the recaustisized chemical recycle stream obtained from the chemical liquor stream 168 by concentrating the chemical liquor stream in a concentration system 166 thereby producing a concentrated chemical liquor stream 164 then burning the concentrated chemical liquor stream in a boiler system 160 thereby producing chemical recycle stream 158 and a flue gas stream 162, then converting the sodium carbonate to sodium hydroxide in the recaustisizing system 156 by contacting with lime (CaO) 152 producing the cooking liquor feed stream 154 containing sodium hydroxide. Digested biomass 112 is obtained from the digestion system 110 by at least partially digesting the lignin, celluloses and hemicelluloses in the predigested biomass. The digested biomass stream 112 is then processed through a wash system 114 that may have one or more washing steps to remove at least a portion of the lignin and residual caustics and sulfur compounds If any. Optionally, water recovered from the concentration system 166 can be recycled as wash water 170 to wash system 114. The thus-lignin removed digested biomass stream (lignin removed digested biomass) 116 may then be provided to other process such as to convert papers, and to produce chemicals and biofuels.

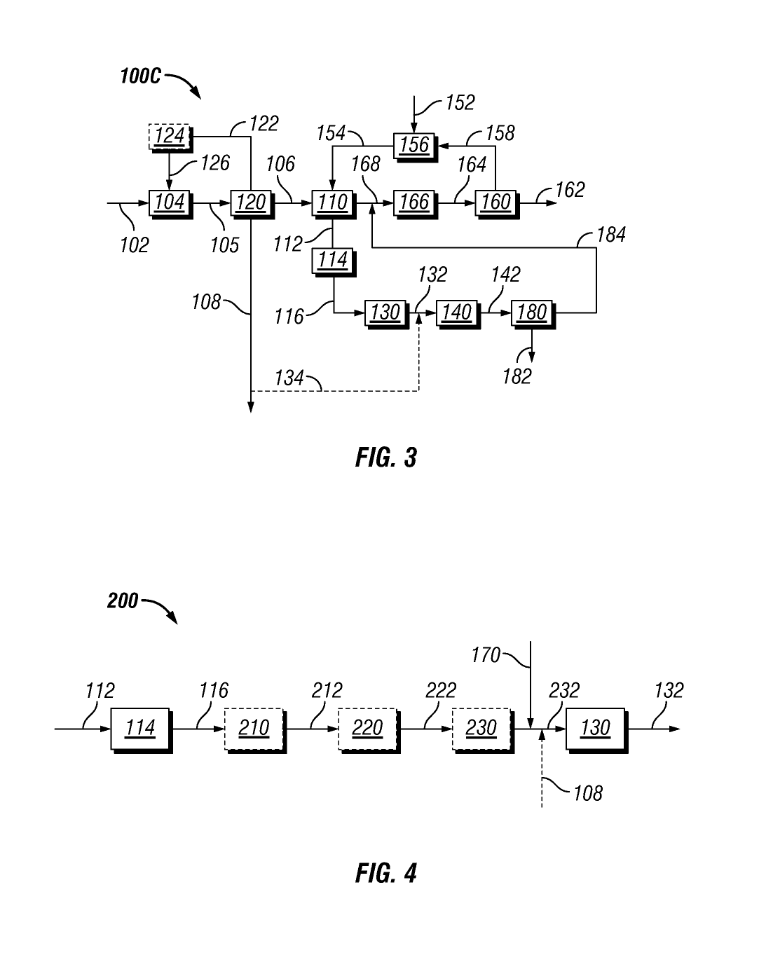

The present process further provides a method of producing an alcohol from a lignocellulosic biomass. In reference to FIG. 2, in another embodiment of the invention process 100B, and to FIG. 3, in yet another embodiment of the invention 100C, biomass 102 is provided to a pretreatment system 104 that may have one or more vessels, where the biomass is contacted with a solution containing at least one α-hydroxysulfonic acid to produce a product stream 105 containing hemicelluloses in solution and a residual biomass containing celluloses and lignin. At least a portion of the solution is separated, in a separation system (and/or acid removal system) 120, from the residual biomass providing a solution stream 108 and a pretreated biomass stream 106. The pretreatment system 104 can comprise a number of components including in situ generated α-hydroxysulfonic acid. The term “in situ” as used herein refers to a component that is produced within the overall process; it is not limited to a particular reactor for production or use and is therefore synonymous with an in process generated component. Optionally the reacted product stream from 104 is introduced to acid removal system 120 where the acid is recovered 122 (and optionally scrubbed 124) and recycled via recycle stream 126 to 104 and product stream 106 containing at least one fermentable sugar (e.g., pentose and optionally hexose) substantially free of the alpha-hydroxysulfonic acids is produced for further processing.

The pretreated biomass stream 106 is contacted, in a digestion system 110 that may have one or more digester(s), with a cooking liquor (optionally via cooking liquor feed stream 154) that was optionally at least a portion recycled from the recaustisized chemical recycle stream obtained from the chemical liquor stream 168 by concentrating the chemical liquor stream in a concentration system 166 thereby producing a concentrated chemical liquor stream 164 then burning the concentrated chemical liquor stream in a boiler system 160 thereby producing chemical recycle stream 158 and a flue gas stream 162, then converting the sodium carbonate to sodium hydroxide in the recaustisizing system 156 by contacting with lime (CaO) 152 producing the cooking liquor feed stream 154 containing sodium hydroxide. Digested biomass 112 is obtained from the digestion system 110 by at least partially digesting the lignin and hemicelluloses in the predigested biomass. The digested biomass stream 112 is then processed through a wash system 114 that may have one or more washing steps. Optionally, water recovered from the concentration system 166 can be recycled as wash water 170 to wash system 114. The thus-lignin removed digested biomass stream 116 is provided to the enzymatic hydrolysis system 130 as feedstock or is then optionally concentrated by mechanical dewatering system 210 (FIG. 4) thereby producing high solids digested biomass stream 212 then provided to the enzymatic hydrolysis system 130. In one preferred embodiment, at least a portion of or the entire solution stream 108 can be provided to the enzymatic hydrolysis system 130 via hydrolysis stream. In the enzymatic hydrolysis system 130, digested biomass and optionally hemicelluloses from the solution stream is hydrolyzed with an enzyme solution, whereby hydrolyzate (aqueous sugar stream) 132 is produced and fermented in the fermentation system 140 in the presence of a microorganism(s) to produce a fermented product stream containing at least one alcohol (alcohol stream 142). In another preferred embodiment, at least a portion of or the entire solution stream 108 can be provided to the fermentation system 140 via fermentation stream 134. The alcohol 182 can then be recovered in a recovery system 180 from the alcohol stream 142 also producing aqueous effluent stream 184. Lignin can be optionally removed after the hydrolysis system, after the fermentation system or after the recovery system by lignin separation system 120, a, b, c, respectively removing lignin as a wet solid residue 128 a, b, c. The aqueous effluent stream after the removal of lignin can be optionally recycled as aqueous effluent recycle stream 186 to the chemical recycle stream 158 thereby reducing fresh water intake in the overall process. Optionally, the aqueous effluent recycle stream 186 can be recycled as wash water to wash system 114. In reference to FIG. 3, in another embodiment of the invention process, in addition to the process described for FIG. 2 above, the aqueous effluent stream 184 can be recycled without the lignin separation system to the chemical liquor stream 168 and recycled and processed as described above. In reference to FIG. 4, in another embodiment of the invention process 200, the lignin removed digested biomass stream 110 is optionally concentrated by mechanical dewatering system 210 thereby producing high solids digested biomass stream 212 then provided to the enzymatic hydrolysis system 130. The lignin removed digested biomass stream 110 or the high solids digested biomass stream 212 is optionally delignified in oxygen delignification system 220 thereby producing delignified digested biomass stream 222 then provided to the enzymatic hydrolysis system 130. In another embodiment, the lignin removed digested biomass stream 110, the high solids digested biomass stream 212, or the delignified digested biomass stream 222 is optionally mechanically refined in mechanical refining system 230 thereby producing a refined digested biomass stream 232 then provided to the enzymatic hydrolysis system 130. Any of 210, 220 or 230 system can be optionally used in any combination of one, two or three process combinations. The Figures are included as an example of how the present invention can be practiced and is not meant to be limiting in any manner.

Any suitable (e.g., inexpensive and/or readily available) type of biomass can be used. Suitable lignocellulosic biomass can be, for example, selected from, but not limited to, forestry residues, agricultural residues, herbaceous material, municipal solid wastes, waste and recycled paper, pulp and paper mill residues, and combinations thereof. Thus, in some embodiments, the biomass can comprise, for example, corn stover, straw, bagasse, miscanthus, sorghum residue, switch grass, bamboo, water hyacinth, hardwood, hardwood chips, hardwood pulp, softwood, softwood chips, softwood pulp, and/or combination of these feedstocks. The biomass can be chosen based upon a consideration such as, but not limited to, cellulose and/or hemicelluloses content, lignin content, growing time/season, growing location/transportation cost, growing costs, harvesting costs and the like.

Prior to pretreatment with the solution, the biomass can be washed and/or reduced in size (e.g., chopping, crushing or debarking) to a convenient size and certain quality that aids in moving the biomass or mixing and impregnating the chemicals from cooking liquor. Thus, in some embodiments, providing biomass can comprise harvesting a lignocelluloses-containing plant such as, for example, a hardwood or softwood tree. The tree can be subjected to debarking, chopping to wood chips of desirable thickness, and washing to remove any residual soil, dirt and the like.

In the pretreatment system, various factors affect the conversion of the biomass feedstock in the hydrolysis reaction. The components of alpha-hydroxysulfonic acids, including carbonyl compound or incipient carbonyl compound (such as trioxane) with sulfur dioxide and water, should be added to in an amount and under conditions effective to form alpha-hydroxysulfonic acids. The temperature and pressure of the hydrolysis reaction should be in the range to form alpha-hydroxysulfonic acids and to hydrolyze biomass into fermentable sugars. The amount of carbonyl compound or its precursor and sulfur dioxide should be to produce alpha-hydroxysulfonic acids in the range from about 1 wt %, preferably from about 5 wt %, most preferably from about 10 wt %, to about 55 wt %, preferably to about 50 wt %, more preferably to about 40 wt %, based on the total solution. For the reaction, excess sulfur dioxide is not necessary, but any excess sulfur dioxide may be used to drive the equilibrium in equation 1 below to favor the acid form at elevated temperatures. The contacting conditions of the hydrolysis reaction may be conducted at temperatures preferably at least from about 50° C. depending on the alpha-hydroxysulfonic acid used, although such temperature may be as low as room temperature depending on the acid and the pressure used. The contacting condition of the hydrolysis reaction may range preferably up to and including about 150° C. depending on the alpha-hydroxysulfonic acid used. In a more preferred condition the temperature is at least from about 80° C., most preferably at least about 100° C. In a more preferred condition the temperature range up to and including about 90° C. to about 120° C. The reaction is preferably conducted at as low a pressure as possible, given the requirement of containing the excess sulfur dioxide. The reaction may also be conducted at a pressure as low as about 1 barg, preferably about 4 barg, to about pressure of as high as up to 10 barg The temperature and pressure to be optimally utilized will depend on the particular alpha-hydroxysulfonic acid chosen and optimized based on economic considerations of metallurgy and containment vessels as practiced by those skilled in the art.

The amount of acid solution to “dry weight” biomass determines the ultimate concentration of fermentable sugar obtained. Thus, as high a biomass concentration as possible is desirable. This is balanced by the absorptive nature of biomass with mixing, transport and heat transfer becoming increasingly difficult as the relative amount of biomass solids to liquid is increased. Numerous methods have been utilized by those skilled in the art to circumvent these obstacles to mixing, transport and heat transfer. Thus weight percentage of biomass solids to total liquids (consistency) may be as low as 1% or as high as 33% depending on the apparatus chosen and the nature of the biomass.

The temperature of the hydrolysis reaction can be chosen so that the maximum amount of extractable carbohydrates are hydrolyzed and extracted as fermentable sugar (more preferably pentose and/or hexose) from the biomass feedstock while limiting the formation of degradation products. The time and temperature of contact is such that effectively produces a pretreated stream containing a solution containing hemicelluloses and a pretreated biomass containing celluloses and lignin. At least a portion of the of the solution is separated from the pretreated stream providing an solution stream containing hemicelluloses and a pre-digested biomass stream containing celluloses and lignin that is further provided to the digestion system. Some lignin or cellulose may be present in the solution stream and some hemicelluloses may be remaining in the pretreated biomass stream. In an embodiment, the solution stream can be recycled to concentrate the hemicelluloses to higher than 10 wt %, preferably even higher than 15 wt % before further processing.

The alpha-hydroxysulfonic acids of the general formula

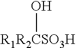

where R₁ and R₂ are individually hydrogen or hydrocarbyl with up to about 9 carbon atoms that may or may not contain oxygen can be used in the treatment of the instant invention. The alpha-hydroxysulfonic acid can be a mixture of the aforementioned acids. The acid can generally be prepared by reacting at least one carbonyl compound or precursor of carbonyl compound (e.g., trioxane and paraformaldehyde) with sulfur dioxide and water according to the following general equation 1.

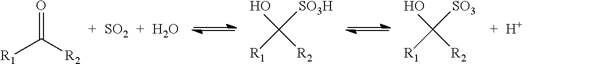

where R₁ and R₂ are individually hydrogen or hydrocarbyl with up to about 9 carbon atoms or a mixture thereof.

Illustrative examples of carbonyl compounds useful to prepare the alpha-hydroxysulfonic acids used in this invention are found where

R₁═R₂═H (formaldehyde)
R₁═H, R₂═CH₃ (acetaldehyde)
R₁═H, R₂═CH₂CH₃ (propionaldehyde)
R₁═H, R₂═CH₂CH₂CH₃ (n-butyraldehyde) R₁═H, R₂═CH(CH₃)₂ (i-butyraldehyde)
R₁═H, R₂═CH₂OH (glycolaldehyde)
R₁═H, R₂═CHOHCH₂OH (glyceraldehdye)
R1=H, R2=C(═O)H (glyoxal)

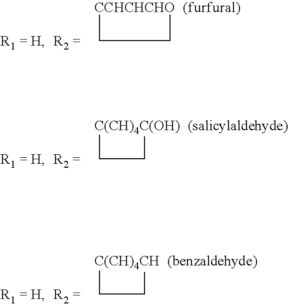

R₁═R₂═CH₃ (acetone)
R₁═CH₂OH, R₂═CH₃ (acetol)
R₁═CH₃, R₂═CH₂CH₃ (methyl ethyl ketone)
R₁═CH₃, R₂═CHC(CH₃)₂ (mesityl oxide)
R₁═CH₃, R₂═CH₂CH(CH₃)₂ (methyl i-butyl ketone)
R₁, R₂═(CH₂)₅ (cyclohexanone) or
R₁═CH₃, R₂═CH₂Cl (chloroacetone)

The carbonyl compounds and its precursors can be a mixture of compounds described above. For example, the mixture can be a carbonyl compound or a precursor such as, for example, trioxane which is known to thermally revert to formaldehyde at elevated temperatures or an alcohol that maybe converted to the aldehyde by dehydrogenation of the alcohol to an aldehyde by any known methods. An example of such a conversion to aldehyde from alcohol is described below. An example of a source of carbonyl compounds maybe a mixture of hydroxyacetaldehyde and other aldehydes and ketones produced from fast pyrolysis oil such as described in “Fast Pyrolysis and Bio-oil Upgrading, Biomass-to-Diesel Workshop”, Pacific Northwest National Laboratory, Richland, Wash., Sep. 5-6, 2006. The carbonyl compounds and its precursors can also be a mixture of ketones and/or aldehydes with or without alcohols that may be converted to ketones and/or aldehydes, preferably in the range of 1 to 7 carbon atoms.

The preparation of α-hydroxysulfonic acids by the combination of an organic carbonyl compounds, SO₂ and water is a general reaction and is illustrated in equation 2 for acetone.

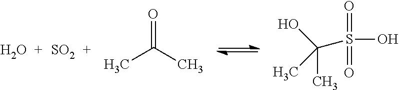

The α-hydroxysulfonic acids appear to be as strong as, if not stronger than, HCl since an aqueous solution of the adduct has been reported to react with NaCl freeing the weaker acid, HCl (see U.S. Pat. No. 3,549,319). The reaction in equation 1 is a true equilibrium, which results in facile reversibility of the acid. That is, when heated, the equilibrium shifts towards the starting carbonyl, sulfur dioxide, and water. If the volatile components (e.g. sulfur dioxide) is allowed to depart the reaction mixture via vaporization or other methods, the acid reaction completely reverses and the solution becomes effectively neutral. Thus, by increasing the temperature and/or lowering the pressure, the sulfur dioxide can be driven off and the reaction completely reverses due to Le Châtelier's principle, the fate of the carbonyl compound is dependant upon the nature of the material employed. If the carbonyl is also volatile (e.g. acetaldehyde), this material is also easily removed in the vapor phase. Carbonyl compounds such as benzaldehyde, which are sparingly soluble in water, can form a second organic phase and be separated by mechanical means. Thus, the carbonyl can be removed by conventional means, e.g., continued application of heat and/or vacuum, steam and nitrogen stripping, solvent washing, centrifugation, etc. Therefore, the formation of these acids is reversible in that as the temperature is raised, the sulfur dioxide and/or aldehyde and/or ketone can be flashed from the mixture and condensed or absorbed elsewhere in order to be recycled. It has been found that these reversible acids, which are approximately as strong as strong mineral acids, are effective in biomass treatment reactions. We have found that these treatment reactions produce very few of the undesired byproducts, furfurals, produced by other conventional mineral acids. Additionally, since the acids are effectively removed from the reaction mixture following treatment, neutralization with base and the formation of salts to complicate downstream processing is substantially avoided. The ability to reverse and recycle these acids also allows the use of higher concentrations than would otherwise be economically or environmentally practical. As a direct result, the temperature employed in biomass treatment can be reduced to diminish the formation of byproducts such as furfural or hydroxymethylfurfural.

It has been found that the position of the equilibrium given in equation 1 at any given temperature and pressure is highly influenced by the nature of the carbonyl compound employed, steric and electronic effects having a strong influence on the thermal stability of the acid. More steric bulk around the carbonyl tending to favor a lower thermal stability of the acid form. Thus, one can tune the strength of the acid and the temperature of facile decomposition by the selection of the appropriate carbonyl compound.

In one embodiment, the acetaldehyde starting material to produce the alpha-hydroxysulfonic acids can be provided by converting ethanol, produced from the fermentation of the treated biomass of the invention process, to acetaldehyde by dehydrogenation or oxidation. Dehydrogenation may be typically carried out in the presence of copper catalysts activated with zinc, cobalt, or chromium. At reaction temperatures of 260-290° C., the ethanol conversion per pass is 30-50% and the selectivity to acetaldehyde is between 90 and 95 mol %. By-products include crotonaldehyde, ethyl acetate, and higher alcohols. Acetaldehyde and unconverted ethanol are separated from the exhaust hydrogen-rich gas by washing with ethanol and water. Pure acetaldehyde is recovered by distillation, and an additional column is used to separate ethanol for recycle from higher-boiling products. It may not be necessary to supply pure aldehdye to the α-hydroxysulfonic acid process above and the crude stream may suffice. The hydrogen-richoff-gas is suitable for hydrogenation reactions or can be used as fuel to supply some of the endothermic heat of the ethanol dehydrogenation reaction. The copper-based catalyst has a life of several years but requires periodic regeneration. In an oxidation process, ethanol maybe converted to acetaldehyde in the presence of air or oxygen and using a silver catalyst in the form of wire gauze or bulk crystals. The reaction is carried out at temperatures between about 500° and about 600° C., depending on the ratio of ethanol to air. Part of the acetaldehyde is also formed by dehydrogenation, with further combustion of the hydrogen to produce water. At a given reaction temperature, the endothermic heat of dehydrogenation partly offsets the exothermic heat of oxidation. Ethanol conversion per pass is typically between 50 and 70%, and the selectivity to acetaldehyde is in the range of about 95 to about 97 mol %. By-products include acetic acid, CO and CO₂. The separation steps are similar to those in the dehydrogenation process, except that steam is generated by heat recovery of the reactor effluent stream. The off-gas steam consists of nitrogen containing some methane, hydrogen, carbon monoxide and carbon dioxide; it can be used as lean fuel with low calorific value. An alternative method to produce acetaldehyde by air oxidation of ethanol in the presence of a Fe—Mo catalyst. The reaction can be carried out at 180-240° C. and atmospheric pressure using a multitubular reactor. According to patent examples, selectivities to acetaldehyde between 95 and 99 mol % can be obtained with ethanol conversion levels above 80%.

In the digestion system, the pretreated biomass is contacted with the cooking liquor in at least one digester where the pretreatment reaction takes place. In one aspect of the embodiment, the cooking liquor contains (i) at least 0.5 wt %, more preferably at least 4 wt %, to 20 wt %, more preferably to 10 wt %, based on the cooking liquor, of at least one alkali selected from the group consisting of sodium hydroxide, sodium carbonate, sodium sulfide, potassium hydroxide, potassium carbonate, ammonium hydroxide, and mixtures thereof, (ii) optionally, 0 to 3%, based on the cooking liquor, of anthraquinone, sodium borate and/or polysulfides; and (iii) water (as remainder of the cooking liquor). In some embodiments, the cooking liquor may have an active alkali of between 5 to 25%, more preferably between 10 to 20%. The term “active alkali” (AA), as used herein, is a percentage of alkali compounds combined, expressed as sodium oxide based on weight of the biomass less water content (dry solid biomass). If sodium sulfide is present in the cooking liquor, the sulfidity can range from about 15% to about 40%, preferably from about 20 to about 30%. The term “sulfidity”, as used herein, is a percentage ratio of Na₂S, expressed as Na₂O, to active alkali. The biomass to cooking liquor ratio can be within the range of 2 to 6, preferably 3 to 5. The digestion reaction is carried out at a temperature within the range of 60° C. to 230° C., and a residence time within 0.25 h to 4 h. The reaction is carried out under conditions effective to provide a digested biomass stream containing digested biomass having a lignin content of 1% to 20% by weight, based on the digested biomass, and a chemical liquor stream containing sodium compounds and dissolved lignin and hemicelluloses material.

The predigester and digester can be, for example, a pressure vessel of carbon steel or stainless steel or similar alloy. The pretreatment system and digestion system can be carried out in the same vessel or in a separate vessel. The cooking can be done in continuous or batch mode. Suitable pressure vessels include, but are not limited to the “PANDIA™ Digester” (Voest-Alpine Industrienlagenbau GmbH, Linz, Austria), the “DEFIBRATOR Digester” (Sunds Defibrator AB Corporation, Stockholm, Sweden), M&D (Messing & Durkee) digester (Bauer Brothers Company, Springfield, Ohio, USA) and the KAMYR Digester (Andritz Inc., Glens Falls, N.Y., USA).

The cooking liquor has a pH from 8 to 14, preferably around 10 to 13 depending on alkali used. The pH of the system may be adjusted from acidic to the pH of the cooking liquor prior to entry of the digestion system, however, it is not necessary to do so and the pretreated biomass stream may be directly contacted with the cooking liquor. The contents can be kept at a temperature within the range of from about 60° C. to about 230° C., preferably from about 100° C. to about 230° C., for a period of time, more preferably within the range from about 130° C. to about 180° C. The period of time can be from about 0.25 to about 4.0 hours, preferably from about 0.5 to about 2 hours, after which the pretreated contents of the digester are discharged. For adequate penetration, a sufficient volume of liquor is required to ensure that all the chip surfaces are wetted. Sufficient liquor is supplied to provide the specified cooking liquor to biomass ratio. The effect of greater dilution is to decrease the concentration of active chemical and thereby reduce the reaction rate.

The invention process has significant benefits over other acidic pretreatments wherein the toxic components such as furfural and acetic acid are essentially eliminated for the fermentation system. Also, bulk removal of lignin allows improved mass transfer of enzymes to cellulose for conversion to fermentable sugars and lower equipment and energy requirements due to smaller volumes going forward. In an embodiment of the process allows for higher recovery of carbohydrates and thereby increased yields. In another embodiment of the process allows additional flexibility to treat a hemicelluloses rich stream to be converted to a fuel or chemical via different processing route more amenable to the chemical composition of this stream. For example, the five-carbon sugars present in the hemicelluloses rich stream can easily be converted to furanic fuels via dehydration in high yields without fermentation that requires long residence times and hence high capital investments. In another embodiment pre-digestion of the hemicelluloses allows to reduce the load on the recovery boiler in the pulp mill, thereby allowing to process increased capacity of feed and hence more fuel.

In some embodiments, the pretreatment could further comprise the use of one or more additives to increase the yield of carbohydrates. Such additives include, but are not limited to, anthraquinone, sodium borate and sodium polysufides and combinations thereof.

In the wash system, the digested biomass stream can be washed to remove one or more of non-cellulosic material, non-fibrous cellulosic material, and non-degradable cellulosic material prior to enzymatic hydrolysis. The digested biomass stream is washed with water stream under conditions to remove at least a portion of lignin and hemicellulosic material in the digested biomass stream and producing lignin removed digested biomass stream having solids content of 5% to 15% by weight, based on the lignin removed digested biomass stream. For example, the digested biomass stream can be washed with water to remove dissolved substances, including degraded, but non-fermentable cellulose compounds, solubilised lignin, and/or any remaining alkaline chemicals such as sodium compounds that were used for cooking or produced during the cooking (or pretreatment). The lignin removed digested biomass stream may contain higher solids content by further processing such as mechanical dewatering as described below.

In a preferred embodiment, the digested biomass stream is washed counter-currently. The wash can be at least partially carried out within the digester and/or externally with separate washers. In one embodiment of the invention process, the wash system contains more than one wash steps, for example, first washing, second washing, third washing, etc. that produces lignin removed digested biomass stream from first washing, lignin removed digested biomass stream from second washing, etc. operated in a counter current flow with the water, that is then sent to subsequent processes as lignin removed digested biomass stream. The water is recycled through first recycled wash stream and second recycled wash stream and then to third recycled wash stream Water recovered from the chemical liquor stream by the concentration system can be recycled as wash water to wash system. It can be appreciated that the washed steps can be conducted with any number of steps to obtain the desired lignin removed digested biomass stream. Additionally, in one embodiment the washing step adjusts the pH for subsequent hydrolysis step where the pH is about 5. In another embodiment the pH of the pulp can be adjust using the CO₂ released from sugars fermentation.

In some embodiments, the materials or chemicals can be regenerated thereby reducing the addition of fresh make-up chemical cost and lowering the load on the effluent plant. The recovery of chemicals and energy from the residual chemical liquor stream are integral part of the process. In one embodiment, a weak chemical liquor stream (about 15% solids), that can be obtained from the digested biomass wash system, from the digestive system, and optionally from a oxygen delignification unit, is concentrated through a series of evaporation and chemical addition steps into a heavy or concentrated chemical liquor at about 60% to about 75% solids. Subsequently, the concentrated chemical liquor stream is incinerated (or burned) in the recovery furnace to form inorganic smelt. The lignin and the solubilised sugar components can be used as an energy source in this combustion step. In some embodiments, lignin collected following an enzymatic hydrolysis step can be optionally added to the concentrated chemical stream to increase the lignin content. In some embodiments, the lignin can be used as energy source to provide heat during the distillation of alcohol or any other step in the biomass-to-alcohol process. In some embodiments, the lignin can be co-fired as fuel for the lime-kiln in the recausticizing operation or in a power boiler for steam and power generation. The smelt from the furnace can be dissolved by addition of water or any recycle aqueous stream (for example, the aqueous effluent stream from bottoms of the distillation). The chemicals are then subjected to recausticizing operation where the chemicals are regenerated using burned lime to form the cooking liquor.

Optionally, the pretreated and washed biomass can be refined using any suitable mechanical refining device to further break down the material in size prior to enzymatic hydrolysis. For example, the contents of the pretreatment pressure vessel can be discharged into a mechanical disc refiner or PFI refiner (or other typical refiner used in the pulping industry) to break the cooked biomass open and reduce the cooked biomass to fibers that have improved enzymatic digestibility. In some embodiments, the refining can provide bundles of cellulose fibers, single cellulose fibers, fragments of cellulose fibers, or combinations thereof. In some embodiments, refining provides largely single fibers and bundles of single fibers. In some embodiments, refining can provide pretreated biomass wherein over 90% of the material is single fibers or fragments of single fibers.

Generally, not all the lignin is removed by the pretreatment reaction. In some embodiments, at least a portion of the residual lignin can be removed from the lignin removed digested biomass stream by oxygen delignification. Accordingly, in some embodiments, solids from the pretreated lignocellulosic mixture can be collected (via filtration or decanting of any liquids), dried and placed in an aqueous alkaline solution (e.g., water comprising 2% to 5% by weight of NaOH). The alkaline solution of solids can then be placed in a pressurized vessel and treated with oxygen gas at an elevated temperature, such as between about 60° C. and about 150° C., for a period of time effective to remove at least a portion of the lignin, such as between about 10 minutes to about 4 hours. In some embodiments, the lignin can then be removed via washing (e.g., in water). In some embodiments, oxygen delignification can be performed prior to a refining system, such that the final pretreated lignocellulosic biomass mixture (i.e., the biomass used for enzymatic hydrolysis and fermentation) is a mixture that has been treated with cooking liquor, washed, subjected to oxygen delignification, and refined. In an oxygen delignification system, a portion of the lignin is removed from one of lignin removed digested biomass stream, hydrolyzate, or alcohol stream prior to step (d) or (e). The resulting lignin removed digested biomass stream, hydrolyzate, or alcohol stream containing less than 5 wt % lignin content, more preferably less than 3 wt % lignin content, based on such stream.

Optionally, the lignin removed digested biomass stream can be concentrated by mechanical dewatering to produce a high solids digested biomass stream having about 15 wt % to about 40 wt % solids. The mechanical dewatering can be carried out by any mechanical dewatering devices including, for example, filter presses, rotary washers and/or screw presses, to produce a high solids digested biomass stream having up to 40 wt % solids, more preferably up to 30 wt % solids. Higher consistency (or solids) digested biomass leads to concentrated beer stream at the back end, thereby lowing the equipment size for the hydrolysis/fermentation vessels reducing the capital cost and additionally saving on energy, e.g. 50% energy saving by distilling concentrated (about 10%) versus dilute beer stream (about 4%).

Therefore, in another embodiment, solids in the lignin removed digested biomass stream is mechanically refined prior to contacting the lignin removed digested biomass stream with cellulases in step (g), thereby reducing the solids in size. In another embodiment, the concentrated lignin removed digested biomass stream is subjected to oxygen delignification prior to contacting the lignin removed digested biomass stream with cellulases in step (j). In another embodiment, the concentrated lignin removed digested biomass stream is subjected to mechanically refining solids in the lignin removed digested biomass stream prior to contacting the lignin removed digested biomass stream with cellulases in step (i), thereby reducing the solids in size. In another embodiment, the concentrated lignin removed digested biomass stream is subjected to oxygen delignification and mechanically refining solids in the lignin removed digested biomass stream prior to contacting the lignin removed digested biomass stream with cellulases in step (j), thereby reducing the solids in size. In another embodiment, the lignin removed digested biomass stream is subjected to oxygen delignification and mechanically refining solids in the lignin removed digested biomass stream prior to contacting the lignin removed digested biomass stream with cellulases in step (j), thereby reducing the solids in size.

In yet another embodiment, in step (i) the water stream is flowing countercurrent to the digested biomass steam.

In yet another embodiment, at least a portion of the lignin is removed from one of lignin removed digested biomass stream, hydrolyzate, or alcohol stream prior to step (j) or (k) thereby providing a lignin removed digested biomass stream, hydrolyzate, or alcohol stream containing less than 5% lignin content based on said stream.

In yet another embodiment, the chemical liquor stream from step (i) is concentrated to produce a concentrated chemical liquor stream, the concentrated chemical liquor stream is burned to produce a chemical recycle stream, the chemical recycle stream is recausticized to produce a cooking liquor feed stream, and the cooking feed stream is recycled to the digester in step (k) as at least a portion of the cooking liquor. In yet a further embodiment, at least a portion of the lignin is removed from the aqueous effluent stream to produce an aqueous effluent recycle stream which is recycled through the chemical recycle stream.

Optionally, following the pretreatment and/or any other desired pretreatment steps (washing, refining, oxygen delignifying, mechanical dewatering), the pretreated biomass and/or fibers can then be subjected to enzymatic hydrolysis for conversion to fermentable sugars. The enzymatic hydrolysis can be carried out at between about 5% and about 15% fiber consistency or at a higher consistency between about 15% to about 40%. In some embodiments, the lignocelluloses-degrading enzymes can be mixed with pretreated mixture at a fiber consistency of about 5% to about 15% for a few minutes (between about 1-20 minutes), thickened using a filter press and allowed to hydrolyze for an additional period of time at the higher fiber consistency. Additional enzymes can be added to the thinned mixture. The term “fermentable sugar” refers to oligosaccharides and monosaccharides that can be used as a carbon source by a microorganism in a fermentation process.

In the enzymatic hydrolysis processes 130 the pH of the pretreated feedstock to the enzymatic hydrolysis is typically adjusted so that it is within a range which is optimal for the cellulose enzymes used. Generally, the pH of the pretreated feedstock is adjusted to a pH from about 3.0 to about 7.0, or any pH there between. For example, the pH may be within a range of about 4.0 to about 6.0, or any pH there between, more preferably between about 4.5 and about 5.5, or any pH there between, or about 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0 or any pH there between. Since the pretreated feedstock is alkaline, an acid such as, for example, sulfuric acid or nitric acid may be used for the pH adjustment.

The temperature of the pretreated feedstock is adjusted so that it is within the optimum range for the activity of the cellulose enzymes. Generally, a temperature of about 15° C. to about 100° C., about 30° C. to about 70° C. preferably or any temperature there between, is suitable for most cellulose enzymes, for example a temperature of 45, 46, 47, 48, 49, 50, 51, 52, 53, 54, 55° C., or any temperature there between. The cellulase enzymes and the β-glucosidase enzyme are added to the pretreated feedstock, prior to, during, or after the adjustment of the temperature and pH of the aqueous slurry after pretreatment. Preferably the cellulase enzymes and the β-glucosidase enzyme are added to the pretreated lignocellulosic feedstock after the adjustment of the temperature and pH of the slurry.

By the term “cellulase enzymes” or “cellulases,” it is meant a mixture of enzymes that hydrolyze cellulose. The mixture may include cellobiohydrolases (CBH), glucobiohydrolases (GBH), endoglucanases (EG), and β-glucosidase. In a non-limiting example, a cellulase mixture may include EG, CBH, and β-glucosidase enzymes. The EG enzymes primarily hydrolyzes cellulose polymer in the middle of the chain to expose individual cellulose chains. There are two types of CBH enzymes, CBHI and CBHII. CBHI and CBHII cleave the reducing and non-reducing end of the cellulose chains ends to produce cellobiose. The conversion of cellobiose to glucose is carried out by the enzyme β-glucosidase. By the term “β-glucosidase”, it is meant any enzyme that hydrolyzes the glucose dimer, cellobiose, to glucose. The activity of the β-glucosidase enzyme is defined by its activity by the Enzyme Commission as EC 3.2.1.21. The β-glucosidase enzyme may come from various sources; however, in all cases, the β-glucosidase enzyme can hydrolyze cellobiose to glucose. The β-glucosidase enzyme may be a Family 1 or Family 3 glycoside hydrolase, although other family members may be used in the practice of this invention. It is also contemplated that the β-glucosidase enzyme may be modified to include a cellulose binding domain, thereby allowing this enzyme to bind to cellulose.

The enzymatic hydrolysis may also be carried out in the presence of one or more xylanase enzymes. Examples of xylanase enzymes that may also be used for this purpose and include, for examples, xylanase 1, 2 (Xyn1 and Xyn2) and β-xylosidase, which are typically present in cellulase mixtures.

The process of the present invention can be carried out with any type of cellulase enzymes, regardless of their source. Non-limiting examples of cellulases which may be used in the practice of the invention include those obtained from fungi of the genera Aspergillus, Humicola, and Trichoderma, Myceliophthora, Chrysosporium and from bacteria of the genera Bacillus and Thermobifida. In an even more preferred aspect, the filamentous fungal host cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

The cellulase enzyme dosage is chosen to convert the cellulose of the pretreated feedstock to glucose. For example, an appropriate cellulase dosage can be about 0.1 to about 40.0 Filter Paper Unit(s) (FPU or IU) per gram of cellulose, or any amount there between, for example, 0.1, 0.5, 1.0, 2.0. 4.0, 6.0, 8.0, 10.0, 12.0, 14.0, 16.0, 18.0, 20.0, 22.0, 24.0, 26.0, 28.0, 30.0, 32.0, 34.0, 36.0, 38.0, 40.0 FPU (or IU) per gram of cellulose, or any amount. The term Filter Paper Unit(s) refers to the amount of enzyme required to liberate 2 mg of reducing sugar (e.g., glucose) from a 50 mg piece of Whatman No. 1 filter paper in 1 hour at 50° C. at approximately pH 4.8.

In practice, the hydrolysis is carried out in a hydrolysis system, which may include a series of hydrolysis reactors. The number of hydrolysis reactors in the system depends on the cost of the reactors, the volume of the aqueous slurry, and other factors. For a commercial-scale alcohol plant, the typical number of hydrolysis reactors may be 1 to 10, more preferably 2 to 5, or any number there between. In order to maintain the desired hydrolysis temperature, the hydrolysis reactors may be jacketed with steam, hot water, or other heat sources. Preferably, the cellulose hydrolysis is a continuous process, with continuous feeding of pretreated lignocellulosic feedstock and withdrawal of the hydrolysate slurry. However, it should be understood that batch processes are also included within the scope of the present invention. In one embodiment, a series of Continuous Stirred-Tank Reactor (CSTR) may be used for a continuous process. In another embodiment Short Contact-Time Reactor (SCTR) along with finishing reactor may be used. A thinning reactor may or may not be included in the hydrolysis system.

The enzymatic hydrolysis with cellulase enzymes produces an aqueous sugar stream (hydrolyzate) comprising glucose, unconverted cellulose and lignin. Other components that may be present in the hydrolysate slurry include the sugars xylose, arabinose, mannose and galactose, the organic acids acetic acid, glucuronic acid and galacturonic acid, as well as silica, insoluble salts and other compounds.

The hydrolysis may be carried out in two or multiple stages in a semi continuous manner (see U.S. Pat. No. 5,536,325, which is incorporated herein by reference), or may be performed in a single stage.

In the fermentation system 140, the aqueous sugar stream is then fermented by one or more than one fermentation microorganism to produce a fermentation broth comprising the alcohol fermentation product. In one embodiment, the aqueous sugar stream sent to fermentation may be substantially free of undissolved solids, such as lignin and other unhydrolyzed components so that the later step of separating the microorganism from the fermentation broth will result in the isolation of mainly microorganism; for example, lignin removal step is carried out at 120a. The separation may be carried out by known techniques, including centrifugation, microfiltration, plate and frame filtration, crossflow filtration, pressure filtration, vacuum filtration and the like.

In the fermentation system, any one of a number of known microorganisms (for example, yeasts or bacteria) may be used to convert sugar to ethanol or other alcohol fermentation products. The microorganisms convert sugars, including, but not limited to glucose, mannose and galactose present in the clarified sugar solution to a fermentation product.

Many known microorganisms can be used in the present process to produce the desired alcohol for use in biofuels. Clostridia, Escherichia coli (E. coli) and recombinant strains of E. coli, genetically modified strain of Zymomonas mobilis such as described in U.S. Application Publication No. 2003/0162271, and U.S. Application Nos. 60/847,813 and 60/847,856 (the disclosures of which are herein incorporated by reference) are some examples of such bacteria. The microorganisms may further be a yeast or a filamentous fungus of a genus Saccharomyces, Kluyveromyces, Candida, Pichia, Schizosaccharomyces, Hansenula, Kloeckera, Schwanniomyces, Yarrowia, Aspergillus, Trichoderma, Humicola, Acremonium, Fusarium, and Penicillium.

In another embodiment, for example, the fermentation may be performed with recombinant yeast engineered to ferment both hexose and pentose sugars to ethanol. Recombinant yeasts that can ferment one or both of the pentose sugars xylose and arabinose to ethanol are described in U.S. Pat. Nos. 5,789,210 and 6,475,768, European Patent Nos. EP 1,727,890, and EP 1,863,901 and WO 2006/096130 the disclosures of which are herein incorporated by reference. Xylose utilization can be mediated by the xylose reductase/xylitol dehydrogenase pathway (for example, WO9742307 A1 19971113 and WO9513362 A1 19950518) or the xylose isomerase pathway (for example, WO2007028811 or WO2009109631). It is also contemplated that the fermentation organism may also produce fatty alcohols, for example, as described in WO 2008/119082 and PCT/US07/011,923, the disclosures of which are herein incorporated by reference. In another embodiment, the fermentation may be performed by yeast capable of fermenting predominantly C6 sugars for example by using commercially available strains such as Thermosacc and Superstart.

Preferably, the fermentation is performed at or near the temperature and pH optima of the fermentation microorganism. For example, the temperature may be from about 25° to about 55° C., or any amount there between. A typical temperature range for the fermentation of sugar to alcohol using microorganisms is between about 25° C. to about 37° C. or any temperature there between, for example from 25, 26, 27, 28, 29, 30, 31, 32, 33, 34, 35, 36, 37° C. or any temperature there between, although the temperature may be higher if the microorganism is naturally or genetically modified to be thermostable. The pH of a typical fermentation employing microorganisms is between about 3 and about 6, or any pH there between, for example, a pH of 3.0, 3.5, 4.0, 4.5, 5.0, 5.5, 6.0, or any pH there between. The dose of the fermentation microorganism will depend on other factors, such as the activity of the fermentation microorganism, the desired fermentation time, the volume of the reactor and other parameters. It will be appreciated that these parameters may be adjusted as desired by one of skill in the art to achieve optimal fermentation conditions.

The sugar stream may also be supplemented with additional nutrients for growth of the fermentation microorganism. For example, yeast extract, specific amino acids, phosphate, nitrogen sources, salts, trace elements and vitamins may be added to the hydrolysate slurry to support growth and optimize productivity of the microorganism.

The fermentation may be conducted in batch, continuous or fed-batch modes, with or without agitation. The fermentation system may employ a series of fermentation reactors.

Preferably, the fermentation reactors are agitated lightly with mixing. In a typical commercial-scale fermentation, the fermentation may be conducted using a series of reactors, such as 1 to 6, or any number there between.

Optionally, the fermentation may be conducted so that the fermentation microorganisms are separated from the fermentation and sent back to the drawing fermentation reaction. This may involve continuously withdrawing fermentation broth from the fermentation reactor and separating the microorganism from this solution by known separation techniques to produce a microorganism slurry. Examples of suitable separation techniques include, but are not limited to, centrifugation, microfiltration, plate and frame filtration, crossflow filtration, pressure filtration, settling, vacuum filtration and the like.

In some embodiment, the hydrolysis system and fermentation system may be conducted in the same vessel. In one embodiment, the hydrolysis can be partially completed and the partially hydrolyzed stream may be fermented. In one embodiment, a simultaneous saccharification and fermentation (SSF) process where hydrolysis system may be run until the final percent solids target is met and then the hydrolyzed biomass may be transferred to a fermentation system.

The fermentation system produces an alcohol stream 142 containing at least one alcohol having 2 to 18 carbon atoms. In the recovery system 180, when the product to be recovered in the alcohol stream is a distillable alcohol, such as ethanol, the alcohol can be recovered by distillation in a manner known to separate such alcohol from an aqueous stream.

The alcohol stream (separated fermentation broth or beer) sent to the distillation is a dilute alcohol solution including unconverted cellulose and residual lignin. It may also contain components added during the fermentation to support growth of the microorganisms, as well as small amounts of microorganism that may remain after separation. The alcohol stream is preferably degassed to remove carbon dioxide and then pumped through one or more distillation columns to separate the alcohol from the other components. The column(s) in the distillation unit is preferably operated in a continuous mode, although it should be understood that batch processes are also encompassed by the present invention. Furthermore, the column(s) may be operated at greater than atmospheric pressure, at less than atmospheric pressure or at atmospheric pressure. Heat for the distillation process may be added at one or more points either by direct steam injection or indirectly via heat exchangers. The distillation unit may contain one or more points either by direct steam injection or indirectly via heat exchangers. The distillation unit may contain one or more separate beer and rectifying columns. In this case, dilute beer is sent to the beer column where it is partially concentrated. From the beer column, the vapor goes to a rectification column for further purification. Alternatively, a distillation column is employed that comprises an integral enriching or rectification section. The remaining water may be removed from the vapor by a molecular sieve resin, by adsorption, or other methods familiar to those of skill in the art. The vapor may then be condensed and denatured.

If the product to be recovered in the alcohol stream is not a distillable alcohol, such as fatty alcohols, the alcohol can be recovered by removal of alcohols as solids or as oils 182 from the fermentation vessel, thus separating from the aqueous effluent stream 184. In such an embodiment, it will be desirable to remove the lignin prior to the fermentation system as described above. In one embodiment, for example, such recovery can be carried out in a manner described in WO 2008/119082 and PCT/US07/011,923 which disclosures are herein incorporated by reference.

While embodiments of the invention are susceptible to various modifications and alternative forms, specific embodiments thereof are shown by way of examples herein described in detail. It should be understood, that the detailed description thereto are not intended to limit the invention to the particular form disclosed, but on the contrary, the intention is to cover all modifications, equivalents and alternatives falling within the spirit and scope of the present invention as defined by the appended claims. The present invention will be illustrated by the following illustrative embodiment, which is provided for illustration only and is not to be construed as limiting the claimed invention in any way.

### ILLUSTRATIVE EXAMPLES

### General Methods and Materials

### General Methods and Materials

In the examples, the aldehyde or aldehyde precursors were obtained from Sigma-Aldrich Co.

Wheat straw and wood having the following components analyzed using standard TAPPI methods (T-249, T-222, T-211) and had the following average composition on a dry basis:

| | Wheat Straw | Wood |
|---|---|---|
| Glucan | 35.9 | 45 |
| Xylan | 20 | 18 |
| Lignin | 23.7 | 26.9 |
| Others | 20. | 10.1 |

### Analytical Methods

### Determination of Oxygenated Components in Aqueous Layer

A sample or standard is analyzed by injection into a stream of a mobile phase that flows though a Bio-rad column (Aminex HPX-87H, 300 mm×7.8 mm). The reverse phase HPLC system (Shimadzu) equipped with both RI and UV detectors and the signals are recorded as peaks on a data acquisition and data processing system. The components are quantified using external calibration via a calibration curves based on injection of know concentrations of the target components. Some of the components were calculated by using single point of standard. The reference samples contained 0.5 wt % Glucose, Xylose and Sorbitol in water

### HPLC Instrument Conditions:

Column: Bio-Rad Aminex HPX-87H (300 mm×7.8 mm)

Flow Rate: 0.6 ml/minute

Column Oven: 30° C.

Injection Volume: 10 μl

UV Detector: @320 NM

RI Detector: mode—A; range—100

Run Time: 70 minute

Mobile Phase: 5 mM Sulfuric Acid in water

Sample is either injected directly or diluted with water first, but makes sure there is no particulars. Pass through the 0.2 μm syringe filter, if there is precipitation in the sample or diluted sample. Samples were analyzed for Glucose, Xylose, Cellobiose, Sorbitol, Formic Acid, Acetic Acid, Arabinose, hydroxymethyl furfural, and Furfural content.

### EXAMPLES

### General Procedure for the Formation of α-Hydroxysulfonic Acids

Aldehydes and ketones will readily react with sulfur dioxide in water to form α-hydroxy sulfonic acids according to the equation 1 above. These reactions are generally rapid and somewhat exothermic. The order of addition (SO₂ to carbonyl or carbonyl to SO₂) did not seem to affect the outcome of the reaction. If the carbonyl is capable of aldol reactions, preparation of concentrated mixtures (>30% wt.) are best conducted at temperatures below ambient to minimize side reactions. We have found it beneficial to track the course of the reaction using in situ Infrared Spectroscopy (ISIR) employing probes capable of being inserted into pressure reaction vessels or systems. There are numerous manufacturers of such systems such as Mettler Toledo Autochem's Sentinal probe. In addition to being able to see the starting materials: water (1640 cm⁻¹), carbonyl (from approx. 1750 cm⁻¹ to 1650 cm⁻¹ depending on the organic carbonyl structure) and SO₂ (1331 cm⁻¹), the formation of the α-hydroxysulfonic acid is accompanied by the formation of characteristic bands of the SO₃⁻ group (broad band around 1200 cm⁻¹) and the stretches of the α-hydroxy group (single to multiple bands around 1125 cm⁻¹). In addition to monitoring the formation of the α-hydroxy sulfonic acid, the relative position of the equilibrium at any temperature and pressure can be readily assessed by the relative peak heights of the starting components and the acid complex. The definitive presence of the α-hydroxy sulfonic acid under biomass hydrolysis conditions can also be confirmed with the ISIR and it is possible to monitor the growth of sugars in the reaction mixture by monitoring the appropriate IR bands.

### Example 1

### Formation of 40% wt. α-hydroxyethane Sulfonic Acid from Acetaldehyde

Into a 12 ounce Lab-Crest Pressure Reaction Vessel (Fischer-Porter bottle) was placed 260 grams of nitrogen degassed water. To this was added 56.4 grams of acetaldehyde via syringe with stirring. The acetaldehyde/water mixture showed no apparent vapor pressure. The contents of the Fischer-Porter bottle were transferred into a chilled 600 ml C276 steel reactor fitted with SiComp IR optics. A single ended Hoke vessel was charged with 81.9 grams of sulfur dioxide was inverted and connected to the top of the reactor. The SO₂ was added to the reaction system in a single portion. The pressure in the reactor spiked to approximately 3 bar and then rapidly dropped to atmospheric pressure as the ISIR indicated the appearance and then rapid consumption of the SO₂. The temperature of the reaction mixture rose approximately 31° C. during the formation of the acid (from 14° C. to 45° C.). ISIR and reaction pressure indicated the reaction was complete in approximately 10 minutes. The final solution showed an infrared spectrum with the following characteristics: a broad band centered about 1175 cm⁻¹ and two sharp bands at 1038 cm⁻¹ and 1015 cm⁻¹. The reactor was purged twice by pressurization with nitrogen to 3 bar and then venting. This produced 397 grams of a stable solution of 40% wt. α-hydroxyethane sulfonic acid (HESA) with no residual acetaldehyde or SO₂. A sample of this material was dissolved in d₆-DMSO and analyzed by ¹³C NMR, this revealed two carbon absorbances at 81.4, and 18.9 ppm corresponding the two carbons of α-hydroxyethane sulfonic acid with no other organic impurities to the limit of detection (about 800:1).

### Examples 2-5

### Long Term Stability Tests of α-hydroxyethane Sulfonic Acid Followed by Reversal and Overhead Recovery of the α-hydroxyethane Sulfonic Acid

Into a 2 liter C276 Parr reactor fitted with in situ IR optics was added 1000 grams of α-hydroxyethane sulfonic acid (HESA, approx. 5 or 10% wt.) prepared by the dilution of a 40% wt. stock solution of the acid with deionized water. Target concentration was confirmed by proton NMR of the starting mixture integrating over the peaks for water and the acid. Pressure integrity of the reactor system and air atmosphere replacement was accomplished by pressurization with nitrogen to 100 psig where the sealed reactor was held for 15 minutes without loss of pressure followed by venting to atmospheric pressure where the reactor was sealed. The reactor was then heated to 90 to 120° C. and held at target temperature for four hours. During this period of time the in situ IR reveals the presence of HESA, SO₂, and acetaldehyde in an equilibrium mixture. The higher temperature runs having the equilibrium shifted more towards the starting components than the lower temperature runs, indicative of a true equilibrium. At the end of four hours the acid reversal was accomplished via opening the gas cap of the reactor to an overhead condensation system for recovery of the acid and adjusting the reactor temperature to 100° C. This overhead system was comprised of a 1 liter jacketed flask fitted with a fiber optic based in situ IR probe, a dry ice acetone condenser on the outlet and the gas inlet arriving through an 18″ long steel condenser made from a core of ¼″ diameter C-276 tubing fitted inside of ½″ stainless steel tubing with appropriate connections to achieve a shell-in-tube condenser draining downward into the recovery flask. The recovery flask was charged with about 400 grams of DI water and the condenser and jacketed flask cooled with a circulating fluid held at 1° C. The progress of the acid reversion was monitored via the use of in situ IR in both the Parr reactor and the overhead condensation flask. During the reversal the first component to leave the Parr reactor was SO₂ followed quickly by a decrease in the bands for HESA. Correspondingly the bands for SO₂ rise in the recovery flask and then quickly fall as HESA was formed from the combination of vaporized acetaldehyde with this component. The reversal was continued until the in situ IR of the Parr reactor showed no remaining traces of the α-hydroxyethane sulfonic acid. The IR of the overheads revealed that the concentration of the HESA at this point had reached a maximum and then started to decrease due to dilution with condensed water, free of α-hydroxyethane sulfonic acid components, building in the recovery flask. The reactor was then sealed and cooled to room temperature. The residual liquid in the Parr reactor and the overhead recovered acid was analyzed via proton NMR for HESA concentration. The results are shown in the table below indicating recovery of acid with virtually no residual HESA in the Parr reactor.

| | Starting | | Reversal | [HESA] | Mass | % of | Overall |
|---|---|---|---|---|---|---|---|
| | [HESA] | Reaction | time | in overhead | overheaded | HESA | Mass |
| Example | % wt. | Temp. ° C. | (min.) | (% wt.) | (g.) | recovered | Balance % |
| 2 | 10.01 | 90 | 42 | 15.15 | 243.1 | 96.9 | 99.4 |
| 3 | 10.07 | 105 | 39 | 14.33 | 241.4 | 91.3 | 99.3 |
| 4 | 5.11 | 105 | 40 | 7.39 | 255.1 | 94.7 | 99.5 |
| 5 | 5.36 | 120 | 37 | 8.42 | 163.3 | 88.5 | 99.4 |

### Example 6

### Pre-Digestion of Wheat Straw with 10% wt. α-hydroxyethane Sulfonic Acid at 120° C. for One Hour Followed by Reversal and Overhead Recovery of the α-hydroxyethane Sulfonic Acid

Into a 2 liter C276 Parr reactor fitted with in situ IR optics was added 120.1 grams of compositional characterized wheat straw [dry basis: xylan 22.1% wt.; glucan 38.7% wt.] chopped to nominal 0.5 cm particles. To this was added 999.1 grams of 9.6% wt. α-hydroxyethane sulfonic acid (HESA) prepared by the dilution of a 40% wt. stock solution of the acid with deionized water. Target concentration of acid was confirmed by proton NMR of the starting mixture integrating over the peaks for water and the acid. The reactor was sealed and the pressure integrity of the reactor system and air atmosphere replacement was accomplished by pressurization with nitrogen to 100 psig where the sealed reactor was held for 15 minutes without loss of pressure followed by venting to atmospheric pressure where the reactor was sealed. The reactor was then heated to 120° C. and held at target temperature for one hour. During this period of time the in situ IR reveals the presence of HESA, SO₂, and acetaldehyde in an equilibrium mixture. At the end of the reaction period the acid reversal was accomplished via opening the gas cap of the reactor to an overhead condensation system for recovery of the acid and adjusting the reactor temperature to 100° C. This overhead recovery system was the same as used in examples 42-45 above. The progress of the acid reversion was monitored via the use of in situ IR in both the Parr reactor and the overhead condensation flask. The reversal was continued for a total of 52 minutes until the in situ IR of the Parr reactor showed no remaining traces of the α-hydroxyethane sulfonic acid or SO₂ in the reaction mixture. The reactor was then sealed and cooled to room temperature. The of overhead condensate added 182.6 grams of mass to the starting water and yielded a 15.0% wt. HESA solution (as analyzed by proton NMR) for a total acid recovery of 91% of the starting HESA employed. The cooled reactor was opened and the contents filtered through a medium glass frit funnel using a vacuum aspirator to draw the liquid through the funnel. The reactor was rinsed with three separate portions of water (noting weight on all rinses, totaling to 754 grams), the rinses being used to complete the transfer of solids and rinse the solids in the funnel. The residual solid was dried to a constant weight in the air and then analyzed for moisture content revealing that approximately 40% of the biomass had dissolved during the acid treatment. HPLC analysis of the 1362 grams of the filtrate plus rinses revealed a recovery of 87.6% of the starting xylan had converted to monomeric xylose and 8.2% of the starting cellulose had converted to glucose. The filtrate and overheads contained negligible amounts of furfural (0.1 grams total). Total material balance of recovered materials to starting materials was 98.2%.

### Example 7-23

The pre-digestion runs with acid and digestion runs with alkali at various operating conditions (indicated in Table 2) were carried out using the same apparatus as Example 6. All the pre-digestion runs are carried out in the presence of α-hydroxyethane sulfonic acid (HESA), while the digestion experiments are carried out in the presence of sodium hydroxide or sodium carbonate as alkali. All the experiments were carried out with biomass to liqour (W:L ratio) ratio as indicated in the Table 1. Pre-digestion runs were carried out with acid recovery procedure as indicated in Example 6. Xylan and Glucan recovery after pre-digestion was obtained using HPLC analysis and feedstock compostion indicated earlier. Yield was calculated as weight percentage ratio of oven dried digested biomass material to the total amount of feed (on dry basis).

TABLE 2
Pre-digestion and Digestion Experiments

| | | | W:L | | Residence | | | | | Yield | Acid | Xylan | Glucan |
|---|---|---|---|---|---|---|---|---|---|---|---|---|---|
| Example | | | weight | Temp | Time | Acid | | Alkali | | (% | recovery | Recovery | Recovery |
| # | Reaction | Substrate | Ratio | [C.] | (min) | Acid | wt % | Alkali | wt % | w/w) | (%) | (%) | (%) |
| 7 | Pre-digestion | wood chips | 1:4 | 120 | 60 | HESA | 10 | | | 77 | 75 | 52 | 4 |
| 8 | Digestion | Ex. 7 | 1:4.5 | 150 | 150 | | | NaOH | 3 | 75 | | | |
| 9 | Pre-digestion | wood chips | 1:10 | 120 | 60 | HESA | 10 | | | 69 | 88 | 78 | 6 |
| 10 | Digestion | Ex. 9 | 1:10 | 150 | 150 | | | NaOH | 3 | 48 | | | |
| 11 | Pre-digestion | wood chips | 1:10 | 100 | 120 | HESA | 10 | | | 72 | 98 | 67 | 2 |
| 12 | Digestion | Ex. 11 | 1:10 | 150 | 150 | | | NaOH | 3 | 53 | | | |
| 13 | Pre-digestion | wood chips | 1:10 | 120 | 120 | HESA | 5 | | | 63 | 89 | 80 | 4 |
| 14 | Digestion | wood chips | 1:4 | 150 | 150 | | | NaOH | 3 | 84 | | | |
| 15 | Digestion | wood chips | 1:10 | 150 | 150 | | | NaOH | 3 | 67 | | | |
| 16 | Pre-digestion | wheat straw | 1:10 | 120 | 60 | HESA | 10 | | | 57 | 94 | 78 | 7.4 |
| 17 | Pre-digestion | wheat straw | 1:10 | 120 | 60 | HESA | 10 | | | 57 | 86 | 76 | 7 |
| 18 | Digestion | Ex. 16 | 1:10 | 150 | 120 | | | NaOH | 3 | 55 | | | |
| 19 | Digestion | Ex. 16 | 1:10 | 120 | 120 | | | NaOH | 3 | 55 | | | |
| 20 | Digestion | Ex. 16 | 1:10 | 150 | 120 | | | NaOH | 1 | 82 | | | |
| 21 | Digestion | Ex. 16 | 1:10 | 110 | 120 | | | Na₂CO₃ | 3 | 83 | | | |
| 22 | Digestion | Ex. 17 | 1:10 | 150 | 120 | | | Na₂CO₃ | 3 | 73 | | | |
| 23 | Digestion | Ex. 17 | 1:10 | 150 | 120 | | | NaOH | 1 | 79 | | | |

### Example 24-34

The digested samples from above-mentioned experiments were subjected to enzymatic hydrolysis using CTec2 (from Novozymes) at 2 different enzymes dosages of 5 mg/g cellulose and 15 mg/g of cellulose. All the enzymatic hydrolysis experiments were carried our at 2 wt % glucan consistency for 72 hrs. Overall hydrolysis for glucan are reported in Table 3 for various substrates. The glucan content of various substrates indicated in Table 3 was obtained using very high enzyme dose (60 mg/g substrate). Glucose conversion indicated was calculated relative to the glucose content measured by high dosage experiments. Total sugar recovery is weight ratio of glucan recovered from enzymatic hydrolysis and xylan/glucan from pre-digestion step by glucan and xylan content of the feedstock (dry basis).

TABLE 3
Enzymatic Hydrolysis Experiments

| | | | Glucose | | Total Sugar | |
|---|---|---|---|---|---|---|
| Example | | Glucose | Conversion (%) | | Recovery (%) | |
| # | Substrate | Content | 5 mg/g | 15 mg/g | 5 mg/g | 15 mg/g |
| 24 | Ex. 8 | 47 | 30 | 56 | 29 | 39 |
| 25 | Ex. 10 | 64 | 39 | 77 | 38 | 50 |
| 26 | Ex. 12 | 68 | 18 | 43 | 27 | 37 |
| 27 | Ex. 13 | 53 | 24 | 50 | 15 | 32 |
| 28 | Ex. 14 | 68 | 32 | 58 | 21 | 38 |
| 29 | Ex. 18 | 60 | 93 | 96 | 61 | 62 |
| 30 | Ex. 19 | 55 | 94 | 100 | 59 | 60 |
| 31 | Ex. 20 | 53 | 62 | 90 | 57 | 69 |
| 32 | Ex. 21 | 53 | 73 | 95 | 62 | 71 |
| 33 | Ex. 22 | 50 | 78 | 100 | 59 | 66 |
| 34 | Ex. 23 | 55 | 63 | 90 | 58 | 69 |

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention.

We claim:

1. A process for producing a digested biomass stream comprising:
(a) providing a biomass containing celluloses, hemicelluloses and lignin;
(b) producing a pretreated stream by contacting the biomass with a solution containing at least one α-hydroxysulfonic acid at a temperature of about 150° C. or less, wherein the pretreated stream comprises a solution comprising at least a portion of hemicelluloses and a residual biomass comprising celluloses and lignin;
(c) providing a solution stream and a pretreated biomass stream by separating at least a portion of the solution from the residual biomass;
(d) providing a digested biomass stream and a chemical liquor stream by contacting the pretreated biomass stream with a cooking liquor comprising (i) about 0.5 wt % to about 20 wt %, based on the cooking liquor, (ii) at least one alkali selected from the group consisting of sodium hydroxide, sodium carbonate, sodium sulfide, potassium hydroxide, potassium carbonate, ammonium hydroxide, and any combination thereof, (iii) water, at a biomass to cooking liquor ratio of 2 to 6, at a temperature from about 60° C. to about 230° C., wherein the digested biomass stream comprises digested biomass containing cellulosic material, hemicellulosic material, and at least a portion of lignin, and the chemical liquor stream comprises at least a portion of lignin and at least one sodium compound, potassium compound, or ammonium compound; and
(e) removing at least a portion of lignin and hemicellulosic material in the digested biomass stream and producing lignin-removed digested biomass stream by washing the digested biomass stream with a water stream.

2. The process of claim 1 further comprising removing the α-hydroxysulfonic acid from the pretreated stream by heating and/or reducing pressure to produce an acid-removed product substantially free of the α-hydroxysulfonic acid and recycling said removed α-hydroxysulfonic acid to step (b) as components or recombined form.

3. The process of claim 2 wherein about 0.1% to about 3%, based on the cooking liquor, of anthraquinone, sodium borate and/or polysulfides is present in the cooking liquor of (d).

4. The process of claim 2 where the cooking liquor has a pH from about 8 to about 14 and a temperature in the range of about 100° C. to about 230° C.

5. The process of claim 2 wherein the cooking liquor comprises about 0.5 wt % to 20 wt %, based on the cooking liquor, of sodium hydroxide.

6. The process of claim 2 wherein the cooking liquor has a sulfidity in the range from about 15% to about 40%.

7. The process of claim 2 wherein the active alkali is the range of about 10% to 20%.

8. The process of claim 2 wherein the cooking liquor to biomass ratio is in the range of about 3 to about 5.

9. The process of claim 1 wherein the α-hydroxysulfonic acid is contacted at a temperature in the range of about 80° C. to about 120° C.

10. A process for producing alcohol comprising:
(a) providing a biomass containing celluloses, hemicelluloses and lignin;
(b) producing a pretreated stream by contacting the biomass with a solution containing at least one α-hydroxysulfonic acid at a temperature of about 150° C. or less, wherein the pretreated stream comprises a solution comprising at least a portion of hemicelluloses and a residual biomass comprising celluloses and lignin;
(c) providing a solution stream and a pretreated biomass stream by separating at least a portion of the solution from the residual biomass;
(d) providing a digested biomass stream and a chemical liquor stream by contacting the pretreated biomass stream with a cooking liquor comprising (i) about 0.5 wt % to about 20 wt %, based on the cooking liquor, (ii) at least one alkali selected from the group consisting of sodium hydroxide, sodium carbonate, sodium sulfide, potassium hydroxide, potassium carbonate, ammonium hydroxide, and any combination thereof, (iii) water, at a biomass to cooking liquor ratio of 2 to 6, at a temperature from about 60° C. to about 230° C., wherein the digested biomass stream comprises digested biomass containing cellulosic material, hemicellulosic material, and at least a portion of lignin, and the chemical liquor stream comprises at least a portion of lignin and at least one sodium compound, potassium compound, or ammonium compound;
(e) removing at least a portion of lignin and hemicellulosic material in the digested biomass stream and producing lignin-removed digested biomass stream by washing the digested biomass stream with a water stream;
(f) produce a hydrolyzate containing from about 4% to about 30% by weight of fermentable sugar by hydrolyzing the lignin-removed biomass stream with an enzyme solution comprising cellulases and optionally xylanases at a pH in a range of about 3 to about 7 at a temperature in a range of about 30° C. to about 90° C.;
(g) producing an alcohol stream containing at least one alcohol having 2 to 18 carbon atoms by fermenting the hydrolyzate in the presence of a microorganism at a temperature in a range of about 25° C. to about 55° C. at a pH in a range of about 4 to about 6; and
(h) recovering at least one of said alcohol from the alcohol stream.

11. The process of claim 10 further comprising:
(i) removing the α-hydroxysulfonic acid from the pretreated stream by heating and/or reducing pressure to produce an acid-removed product substantially free of the α-hydroxysulfonic acid and recycling said removed α-hydroxysulfonic acid to step (b) as components or recombined form.

12. The process of claim 10 further comprising providing at least a portion of the solution stream from step (c) to the hydrolyzate prior to fermenting in step (g).

13. The process of claim 10 further comprising providing at least a portion of the solution stream from step (c) to the lignin-removed digested biomass stream prior to hydrolyzing in step (f).

14. The process of claim 10 further comprising concentrating the lignin removed digested biomass stream from step (e) by mechanical dewatering prior to contacting the lignin removed digested biomass stream with cellulases in step (f) thereby increasing the solids content of the lignin removed digested biomass stream from about 15 wt % to about 40 wt % solids.

15. The process of claim 11 further comprising:
(j) produce a concentrated chemical liquor stream by concentrating the chemical liquor stream from step (d);
(k) producing a chemical recycle stream by burning said concentrated chemical liquor stream;
(l) producing a cooking liquor feed stream by recausticizing said chemical recycle stream to; and
(m) recycling the cooking feed stream to the digester in step (d) as at least a portion of the cooking liquor.
